(19) United States
(12) Patent Application Publication
(10) Pub. No.: US 2009/0175952 A1
(43) Pub. Date: Jul. 9, 2009

(54) Cefuroxime axetil granule and process for the preparation thereof

(76) Inventors: Jong Soo Woo, Kyungki-do (KR); Hee Chul Chang, Kyungki-do (KR); Hong Gi Yi, Kyungki-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/584,919

(22) PCT Filed: Jan. 10, 2005
(86) PCT No.: PCT/KR05/00066

§ 371 Date: Jun. 29, 2006

(30) Foreign Application Priority Data
Jan. 9, 2004 (KR) 10-2004-0001597
Aug. 26, 2004 (KR) 10-2004-0067569

(51) Int. Cl. A61K 31/546; A61K 9/16; A61P 31/00

(52) U.S. Cl. 424/497; 514/202

(57) ABSTRACT

A cefuroxime axetil granule composition comprising a non-crystalline cefuroxime axetil solid dispersion or a substantially amorphous cefuroxime axetil, sucrose fatty acid ester, methacrylic acid-ethylacrylate copolymer and a disintegrating agent has highly desirable performance characteristics in terms of masking the bitterness of cefuroxime axetil, as well as high bioavailability and stability of cefuroxime axetil, and thus, can be advantageously used for oral administration of cefuroxime axetil.

### FIELD OF THE INVENTION

The present invention relates to a cefuroxime axetil granule for oral administration having the bitterness of cefuroxime axetil well masked and showing high bioavailability of cefuroxime axetil, and a preparation thereof.

### BACKGROUND OF THE INVENTION

Cefuroxime axetil (CA) is a cephalosporin antibiotic for oral administration having high activity against a wide spectrum of Gram positive and negative microbes. It shows polymorphism of three forms: a crystalline form having a melting point of about 180° C., a substantially amorphous form having a melting point of about 135° C. and a substantially amorphous form having a lower melting point in the range of about 70 to 95° C. The crystalline form of cefuroxime axetil has excellent antibacterial activity, but is only slightly soluble in water and is not readily absorbable in the gastrointestinal tract.

Accordingly, the present inventors had prepared a non-crystalline cefuroxime axetil solid dispersion as disclosed in Korean Patent No. 342943 having an enhanced water-solubility and high bioavailability of cefuroxime axetil.

Further, cefuroxime axetil tastes so bitter that its bitterness cannot be masked with a conventional sweetener or flavoring agent, which causes problems when orally administered to children.

Korean Patent Publication No. 1995-0009097 of GlaxoSmithKline (GSK) discloses a method for preparing granules to mask the bitterness of cefuroxime axetil, comprising the steps of: dispersing the drug in molten stearic acid, spray-drying the resulting dispersion, and cooling the dried product using a low temperature air current. However, the granules obtained by the above process do not dispersed well in water in the formulation process due to the presence of stearic acid, and bitter aftertaste of the formulation still remains causing difficulties when it is orally administrated to people, especially infants.

Accordingly, the present inventors have endeavored to solve the problems associated with the conventional cefuroxime axetil preparation and succeeded in developing an improved cefuroxime axetil granule composition for oral administration which has little bitter taste, high stability and bioavailability of cefuroxime axetil.

### SUMMARY OF THE INVENTION

It is an object of the present invention to provide a cefuroxime axetil granule composition for oral administration having highly desirable performance characteristics in terms of masking the bitterness of cefuroxime axetil as well as high bioavailability and stability of cefuroxime axetil.

It is another object of the present invention to provide a method for preparing a cefuroxime axetil granule using said composition.

In accordance with one aspect of the present invention, there is provided a cefuroxime axetil granule composition comprising a non-crystalline cefuroxime axetil solid dispersion or a substantially amorphous cefuroxime axetil, sucrose fatty acid ester, methacrylic acid-ethylacrylate copolymer, and a disintegrating agent.

In accordance with another aspect of the present invention, there is provided a process for preparing a cefuroxime axetil granule comprising the steps of:

1) mixing sucrose fatty acid ester and methacrylic acid-ethylacrylate copolymer, followed by melting the mixture with heating;

2) dispersing a disintegrating agent and a non-crystalline cefuroxime axetil solid dispersion or a substantially amorphous cefuroxime axetil in the molten mixture obtained in step 1); and

3) cooling the dispersion obtained in step 2), followed by pulverizing the cooled dispersion to obtain the granules.

### BRIEF DESCRIPTION OF THE DRAWINGS

The above and other objects and features of the present invention will become apparent from the following description of the invention, when taken in conjunction with the accompanying drawings, which respectively show:

FIGS. 1(a) to 1(c): differential scanning calorimetry (DSC) scans of sucrose fatty acid ester, methacrylic acid-ethylacrylate copolymer and a molten mixture thereof, respectively;

FIG. 2: the time-dependent changes in the amount of CA leached out of the inventive CA preparation and a comparative preparation (Zinnat® dried syrup, GSK), in distilled water;

FIG. 3: the time-dependent changes in the amount of CA released from the inventive CA preparation and a comparative preparation (Zinnat® dried syrup, GSK), in buffers; and

FIG. 4: the time-dependent plasma levels of CA after administering the inventive CA preparation and a comparative preparation (Zinnat® dried syrup, GSK).

### DETAILED DESCRIPTION OF THE INVENTION

The inventive cefuroxime axetil (CA) granule composition comprises a non-crystalline cefuroxime axetil solid dispersion or a substantially amorphous cefuroxime axetil, a sucrose fatty acid ester, a methacrylic acid-ethylacrylate copolymer and a disintegrating agent as essential components; and may further comprise a coating material and/or a pharmaceutically acceptable additive.

The respective components of the orally administrable granule composition of the present invention are described as follows.

### (1) Cefuroxime Axetil

In the present invention, cefuroxime axetil is used as an active ingredient. Preferred are a non-crystalline cefuroxime axetil solid dispersion prepared by the method disclosed in Korean Patent No. 342943, or a substantially amorphous cefuroxime axetil. These taste as bitter as a crystalline cefuroxime axetil but have better water-solubility and in vivo bioavailability.

### (2) Sucrose Fatty Acid Ester

In the inventive granule composition, the sucrose fatty acid component serves to mask the bitterness of CA. In case of using a sucrose fatty acid ester having a low melting point of 58 to 70° C., the granule composition can melt at a low temperature, which makes the whole process for preparing a CA granule easy. Such a sucrose fatty acid ester is a wax type having some oily characteristics and it plays the role of preventing the drug from leaching out into an aqueous medium.

Preferable examples of the sucrose fatty acid ester include a commercially available SUCROSE F.A.ESTER® (DK ES. F-20W, Dai-ichi Kogyo Seiyaku Inc., Japan), which is a fatty carrier having an HLB (Hydrophilic Lipophilic Balance) value of about 2 and a melting point of about 65 to 68° C.

The sucrose fatty acid ester may be used in an amount of 0.2 to 40 parts by weight, preferably 0.5 to 10 parts by weight, based on 1 part by weight of cefuroxime axetil.

### (3) Methacrylic Acid-Ethylacrylate Copolymer

In the present invention, the methacrylic acid-ethylacrylate copolymer does not melt by itself, but can melt when it is combined with a sucrose fatty acid ester at a mixture ratio of about 1:0.5-1:1.5 by weight, and thus, it may be employed to coat the particles of the drug component into certain type of granule.

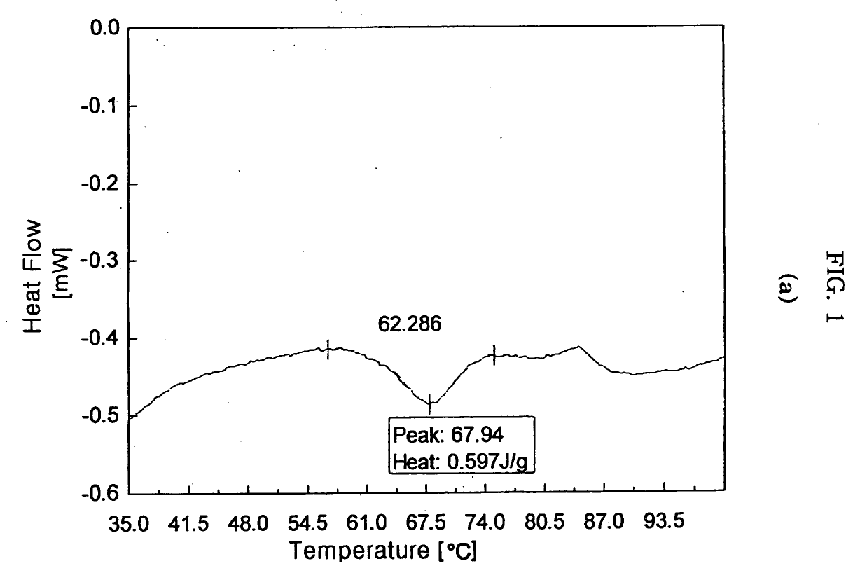

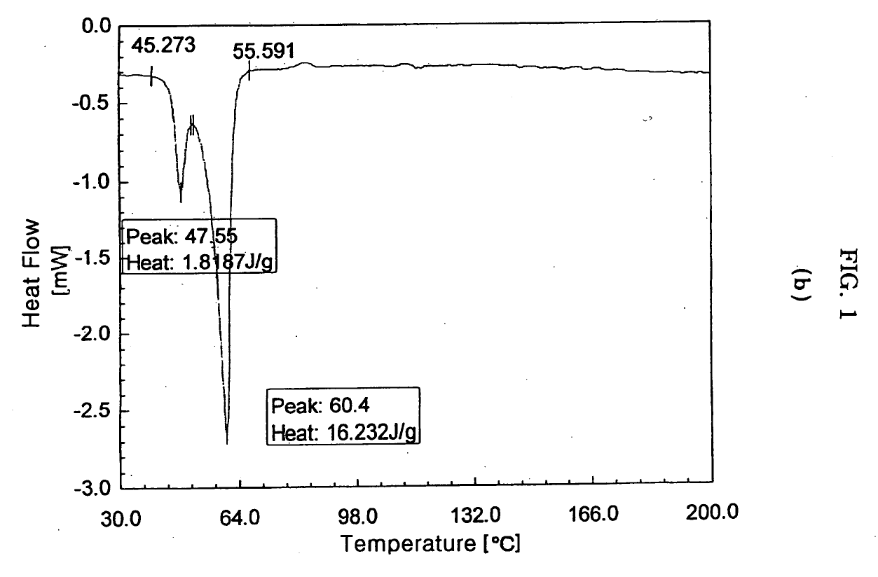

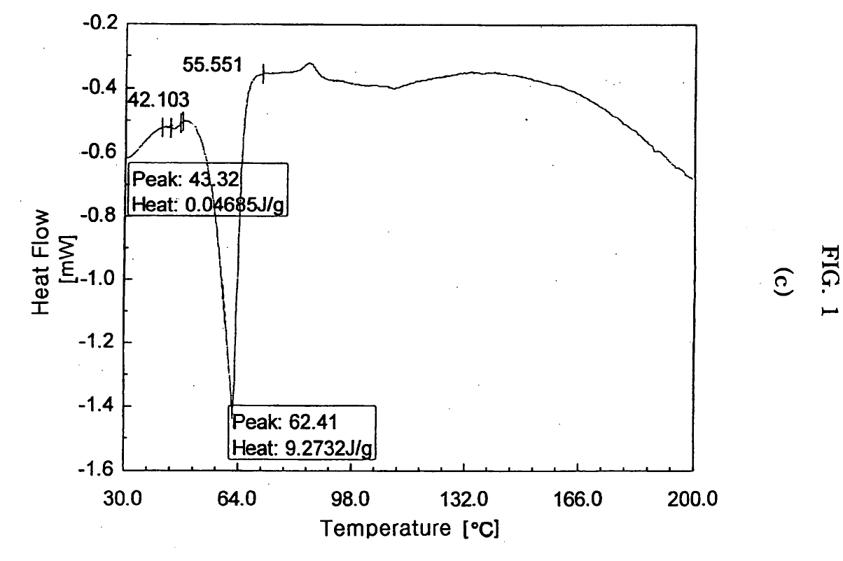

FIGS. 11(a) to 1(c) show DSC scans of a sucrose fatty acid ester, a methacrylic acid-ethylacrylate copolymer and a mixture thereof (a mix ratio of 1:1 by weight), respectively. As shown in FIG. 1(C), when the sucrose fatty acid ester and methacrylic acid-ethylacrylate copolymer are mixed together by melting, a single absorption peak appears on its DSC scan, which is indicative of eutectic melting. Further, the methacrylic acid-ethylacrylate copolymer, an enteric material, easily disintegrates at a pH of 5.5 or more and, thus, plays a role in enhancing the dissolution of the drug.

The methacrylic acid-ethylacrylate copolymer mentioned above is sold by Röhm Inc. under the trade name of Eudragit® L100-55.

The methacrylic acid-ethylacrylate copolymer may be used in an amount of 0.1 to 30 parts by weight, preferably 0.5 to 10 parts by weight, based on 1 part by weight of cefuroxime axetil.

### (4) Disintegrating Agent

The disintegrating agent prompts the disintegration of the inventive granule so that a desired dissolution rate of the drug can be achieved. Representative examples of the disintegrating agent include:

1) fine crystalline cellulose;

2) cross-linked sodium carboxymethyl cellulose;

3) cross-linked polyvinyl pyrrolidone;

4) ion exchange resin, preferably amberlite IRP-88;

5) alginic acid; and

6) sodium starch glycolate.

The above-mentioned disintegrating agent can be used alone or in combination, and most preferred is alginic acid.

The disintegrating agent may be used in an amount of 0.05 to 20 parts by weight, preferably 0.1 to 10 parts by weight, based on 1 part by weight of cefuroxime axetil.

### (5) Coating Material

The inventive granule coated with sucrose fatty acid ester and methacrylic acid-ethylacrylate copolymer can be further coated with an appropriate coating material, if necessary, by a method conventionally used in the art. Preferable coating material may be an enteric material for the protection of cefuroxime axetil.

Representative examples of the enteric coating material include hydroxypropyl methylcellulose phthalate, hydroxypropyl methylcellulose acetate succinate, polyvinyl acetate phthalate, cellulose acetate phthalate, shellac, methacrylic acid-methylmethacrylate copolymer, methacrylic acid-ethylacrylate copolymer. The above-mentioned enteric coating material can be used alone or in combination.

The coating material may be used in an amount of 0.2 to 20 parts by weight, preferably 0.2 to 10 parts by weight, based on 1 part by weight of cefuroxime axetil.

### (6) Pharmaceutically Acceptable Additive

The inventive granule composition may be formulated into various pharmaceutical preparations for oral administration, e.g., in the form of a powder, dried syrup and granule, in accordance with any of the conventional procedure. In order to facilitate the formulation thereof, other suitable pharmaceutically acceptable additive may be added. The additive can be a sweetener such as sugar, a viscosity controlling agent such as gum, emulsifier, a pH controller and a powder excipient for use with powders. Aromatics, coloring agents and flavorings may also be added.

The amount of the pharmaceutically acceptable additive may be 0.01 to 100 parts by weight, preferably 0.02 to 50 parts by weight, based on 1 part by weight of cefuroxime axetil.

A cefuroxime axetil granule having the inventive composition can be prepared by dispersing the disintegrating agent and the non-crystalline cefuroxime axetil solid dispersion or substantially amorphous cefuroxime axetil in a molten mixture of the sucrose fatty acid ester and methacrylic acid-ethylacrylate copolymer, and mixing the resulting dispersion.

A process for preparing a cefuroxime axetil granule having the inventive composition comprises the steps of:

1) mixing the sucrose fatty acid ester and methacrylic acid-ethylacrylate copolymer, followed by melting the mixture with heating;

2) dispersing the disintegrating agent, and the non-crystalline cefuroxime axetil solid dispersion or substantially amorphous cefuroxime axetil in the molten mixture obtained in step 1); and

3) cooling the dispersion obtained in step 2), followed by pulverizing the cooled dispersion to obtain the granule.

It is preferable that the melting process in step 1) is conducted at a temperature ranging from 60 to 75° C. To use the granule prepared above in the form of an orally administrable suspension, it is preferable that the size thereof in step 3) is controlled at 35 mesh or less.

The inventive cefuroxime axetil granule prepared in accordance with the above method effectively masks the bitterness of cefuroxime axetil, and shows high stability and bioavailability of cefuroxime axetil.

The inventive granule may be further coated or formulated into various pharmaceutical forms in combination with other pharmaceutically acceptable carriers, in accordance with any of the conventional procedures.

The following Examples are intended to further illustrate the present invention without limiting its scope.

### EXAMPLE 1

### Preparation of a CA Granule

### 1-1) Preparation of a Non-Crystalline CA Solid Dispersion

100 parts by weight of crystalline cefuroxime axetil (HANMI Fine Chemical Co., Ltd, South Korea) and 16.63 parts by weight of Twin 80® (ICI Inc., USA) were dissolved in acetone, and 16.63 parts by weight of silica was 25 dispersed therein. The dispersion was subjected to spray drying using a spray dryer (Minispray dryer B-191, Buchi, Switzerland) set at an inlet temperature of 45° C. and outlet temperature of 37° C. to obtain a solid dispersion. The solid dispersion was further dried at 30 to 40° C. for about 3 hours to remove residual solvent.

1-2) Preparation of a CA Granule 227 g of sucrose fatty acid ester (sucrose fatty acid ester 37318-31-3, Dai-ichi Kogyo Seiyaku Inc., Japan) as a nonionic surfactant and 318 g of Eudragit® L100-55 (Röhm Inc., USA) were mixed together and the resulting mixture was melted at a temperature of about 75° C. Then, 31.8 g of triacetin as a plasticizer was added thereto, and the resulting molten mixture was agitated and cooled. Before the mixture was completely hardened, 181.8 g of the non-crystalline cefuroxime axetil solid dispersion obtained in Example 1-1) and 45.4 g of alginic acid (Kelacid®, ISP Inc., USA) were dispersed uniformly therein. After fully cooled, the hardened dispersion was pulverized into granules of 35 mesh or less, to obtain 804 g of cefuroxime axetil granules.

### EXAMPLE 2

### Preparation of a CA Granule

The procedure of Example 1-2) was repeated except that a substantially amorphous cefuroxime axetil (Orchid Chemicals & Pharmaceuticals Inc., India) instead of a non-crystalline cefuroxime axetil solid dispersion was used, to prepare cefuroxime axetil granules.

### EXAMPLE 3

### Preparation of a CA Granule

The procedure of Example 1-2) was repeated except that cross-linked sodium carboxymethyl cellulose (AVEBE Inc., USA) instead of alginic acid was used as a disintegrating agent, to prepare cefuroxime axetil granules.

### EXAMPLE 4

### Preparation of a CA Granule

The procedure of Example 1-2) was repeated except that sodium starch glycholate (Penwest Inc., USA) instead of alginic acid was used as a disintegrating agent, to prepare cefuroxime axetil granules.

### EXAMPLE 5

### Preparation of a Coated CA Granule

A coating solution composed of 268 g (80.4 g on a dry-basis) of Eudragit® L30D-55 (Röhm Inc., USA), 8.04 g of Triacetin as a plasticizer and 536 g of distilled water was bottom sprayed to a fluidized bed layer of 804 g of cefuroxime axetil granules prepared in Example 1 using NQ-160 (DALTON Inc., Japan) solution. The coating conditions were as follows: an inlet temperature of 36 to 39° C.; an outlet temperature of 24 to 28° C.; an injection rate of 0.7 to 0.8 ml/minute; and a spraying air pressure of 40 to 50 psi. As a result, 892.4 g of granules coated with Eudragit® L30D-55 and methacrylic acid-methylmethacrylate copolymer were prepared.

### EXAMPLE 6

### Preparation of a Coated CA Granule

The procedure of Example 5 was repeated except that hydroxypropyl methylcellulose phthalate (Shin-Etsu Inc., Japan) was used as a coating material, to prepare 892.4 g of coated granules.

### EXAMPLE 7

### Preparation of a Coated CA Granule

The procedure of Example 5 was repeated except that Eudragit® E-100 (Röhm Inc., USA, a butyl methacrylate-(2-dimethyl aminoethyl) methacrylate-methylmethacrylate copolymer) was used as a coating material, to prepare 892.4 g of coated granules.

### EXAMPLE 8

### Preparation of a Coated CA Granule

The procedure of Example 5 was repeated except that ethyl cellulose (IPI Inc., USA) was used as a coating material, to prepare 892.4 g of coated granules.

### FORMULATION EXAMPLE 1

### Preparation of a Dried Syrup

3022.4 g of sucrose powder, 2.1 g of corn starch, 54.0 g of acesulfame-potassium, 72 g of aspartame and 354.5 g of tutti-frutti flavor® (DaeDo Co. LTD., Korea) were added to 892.4 g of the coated cefuroxime axetil granules prepared in Example 5 and the resulting mixture was mixed thoroughly. Then, 20.5 g of citric acid and 21.6 g of sodium citrate were added thereto and the resulting mixture was mixed together, to prepare a dried syrup of cefuroxime axetil for oral administration.

### FORMULATION EXAMPLE 2

### Preparation of a Dried Syrup

2.1 g of xanthan gum, 14 g of corn starch, 1.4 g of sodium laurylsulfate, 7 g of methyl cellulose, 3012 g of sucrose powder were added to 857.7 g of coated cefuroxime axetil granules prepared in Example 6 and the resulting mixture was mixed thoroughly. Then, 284 g of tutti-frutti flavor®, 284 g of drink flavor powder® (SamYoung Chemical Co. LTD., Korea), 21 g of citric acid and 21.8 g of sodium citrate were added thereto and the resulting mixture was mixed together, to prepare a dried syrup of cefuroxime axetil for oral administration.

### TEST EXAMPLE 1

### Stability Test in an Aqueous Medium

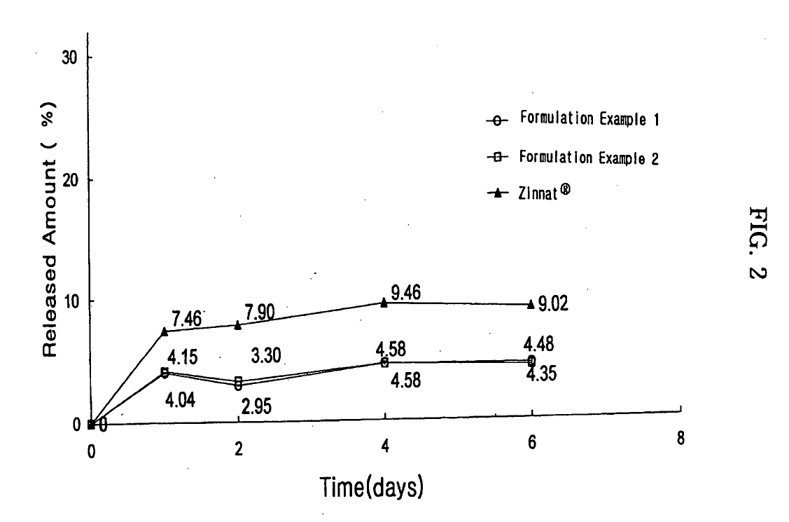

The preparations of Formulation Examples 1 and 2, and commercially available Zinnat® dried syrup (GSK) as a comparative preparation were each suspended in 5 ml of distilled water in an amount corresponding to 150 mg of cefuroxime axetil. The amount of cefuroxime axetil leached out in the distilled water was measured with a UV detector at 278 nm on days 1, 2, 4 and 6. The results are shown in FIG. 2, the released amount of cefuroxime axetil being shown as a relative value (%) based on the initial amount.

As shown in FIG. 2, the inventive preparation exhibited higher stability of cefuroxime axetil in an aqueous medium than the comparative preparation in the actual ready-for-use form.

### TEST EXAMPLE 2

### Dissolution Test

The preparations of Formulation Examples 1 and 2, and commercially available Zinnat® dried syrup (GSK) as a comparative preparation were each subjected to a dissolution test using an amount corresponding to 150 mg of cefuroxime axetil in accordance with the 2^{nd} dissolution test method described in Korean Pharmacopoeia 7^{th} edition under the following conditions:
Test solution: 900 ml of 0.05 mol/l potassium dihydrogen phosphate buffer (pH 7.0)
Temperature of test solution: 37±0.5° C.
Rotation speed: 100 rpm
Detector: UV 278 nm

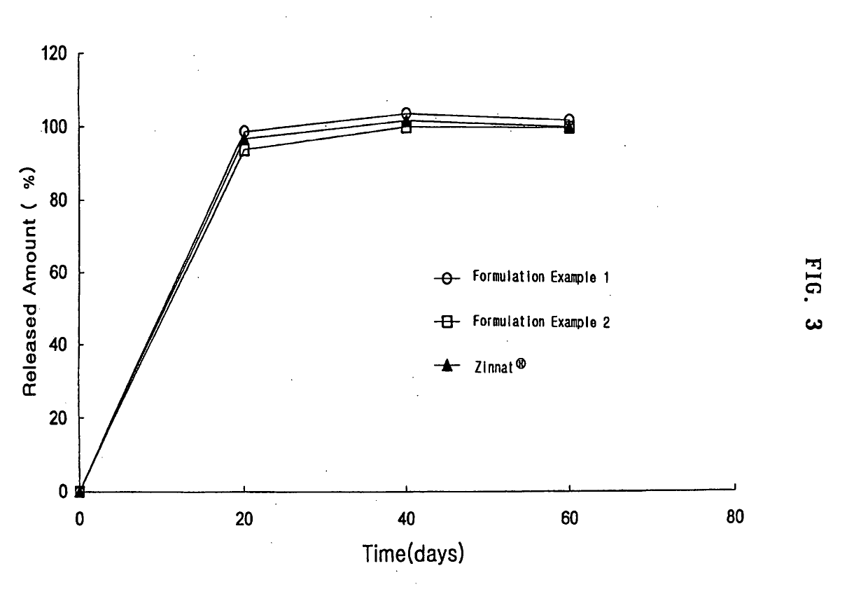

The results are shown in FIG. 3.

As shown in FIG. 3, the inventive preparation shows as good a dissolution property as the comparative preparation.

### TESTING EXAMPLE 3

### Effectiveness in Masking Bitter Taste

The preparations of Formulation Examples 1 and 2, and commercially available Zinnat® dried syrup (GSK) as a comparative preparation were each subjected to sensory evaluation test using an amount corresponding to 150 mg of cefuroxime axetil to check its effectiveness in masking the bitterness of cefuroxime axetil.

Specifically, each of the CA dried syrups of Formulation Examples 1, 2 and Zinnat® dried syrup (GSK) was suspended in 5 ml of distilled water in an amount corresponding to 150 mg of cefuroxime axetil to obtain a syrup therefrom. Test was conducted by having five men and five women, aged 20-30, keep the syrup in the mouth for 10 seconds before spitting out. The strength of bitter taste was recorded for the initial stage (right after spitting) and aftertaste stage (1 minutes after spitting). The results are shown in Table 1.

In Table 1, symbol “A” shows that the number of people who experienced bitter taste is 0-2, while those in “B”, “C” and “D” are 3-5, 6-8 and 9-10, respectively.

TABLE 1

| | Initial stage | Aftertaste stage |
|---|---|---|
| Formulation Example 1 | A | A |
| Formulation Example 2 | A | A |
| Zinnat ® | A | C |

As shown in Table 1, the inventive preparation is superior to the comparative preparation in masking the bitterness of cefuroxime axetil.

### TEST EXAMPLE 4

### Absorption Test

In order to examine the bioavailability of cefuroxime axetil of the inventive preparation, an in vivo absorption test was carried out as follows by employing the preparation of Formulation Example 1, and commercially available Zinnat® dried syrup (GSK) as a comparative preparation. Each preparations was dispersed in 2 ml of water and orally administered to Sprague-Dawley (SD) rats via sonde in an amount corresponding to 20 mg/kg of cefuroxime axetil. Blood samples were taken from the rats 30, 60, 120, 180, 300 and 420 minutes after the administration. The blood samples were treated and analyzed by liquid chromatography by the method disclosed in J. Kor. Pharm. Sci., Vol. 29, No. 4, pages 361-365(1999).
Column: Inertsil ODS-2 (4.6×250 mm) C₁₈
Mobile Phase: 0.05 M ammonium phosphate buffer (pH 3.2):acetonitrile=86:14(v:v)
Injection volume: 50 μl
Flow rate: 1.0 ml/min.
Detector: UV 280 nm

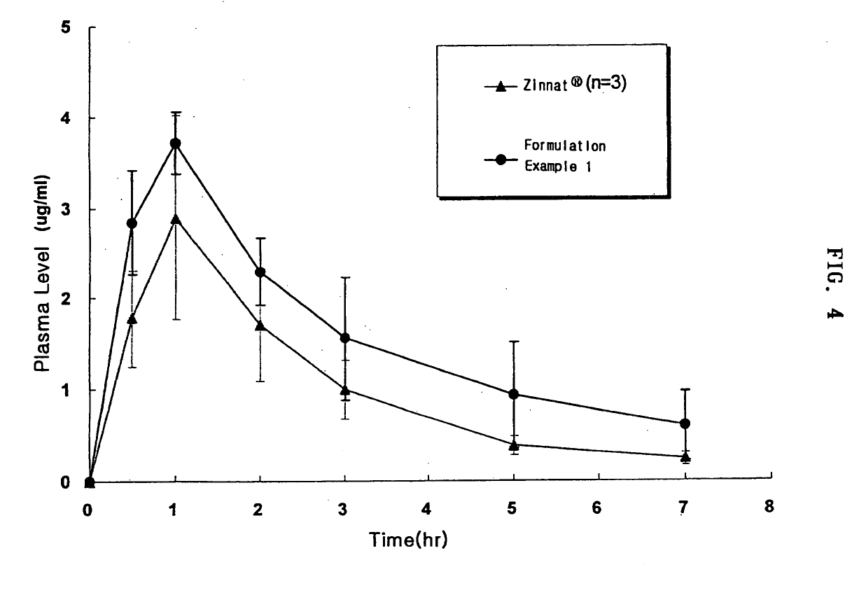

The results are shown in FIG. 4 and Table 2.

TABLE 2

| | AUC7 h (μg · hr/ml)^{*1} | Tₘₐₓ (hr)^{*2} | Cₘₐₓ (μg/ml)^{*3} |
|---|---|---|---|
| Formulation | 11.3 ± 2.5 | 1.0 ± 0.0 | 3.7 ± 0.3 |
| Example 1 | | | |
| Zinnat ® | 7.3 ± 2.5 | 1.0 ± 0.0 | 2.9 ± 1.1 |

^{*1}Area under the plasma CA concentration up to 7 hours after the administration
^{*2}Time at the maximum plasma CA concentration
^{*3}Maximum plasma CA concentration

As shown in FIG. 4 and Table 2, the bioavailability of CA in the inventive preparation is much higher than that of the comparative preparation.

While the invention has been described with respect to the above specific embodiments, it should be recognized that various modifications and changes may be made to the invention by those skilled in the art which also fall within the scope of the invention as defined by the appended claims.

What is claimed is:

1. A cefuroxime axetil granule composition comprising a non-crystalline cefuroxime axetil solid dispersion or a substantially amorphous cefuroxime axetil, a sucrose fatty acid ester, a methacrylic acid-ethylacrylate copolymer, and a disintegrating agent.

2. The cefuroxime axetil granule composition of claim 1, wherein the disintegrating agent is selected from the group consisting of fine crystalline celluloses, cross-linked sodium carboxymethyl celluloses, cross-linked polyvinyl pyrrolidones, ion exchange resins, alginic acid, sodium starch glycolate and a mixture thereof.

3. The cefuroxime axetil granule composition of claim 1, wherein the amounts of the sucrose fatty acid ester, the methacrylic acid-ethylacrylate copolymer and the disintegrating agent are 0.5 to 10, 0.5 to 10, and 0.1 to 10 parts by weight, respectively, based on 1 part by weight of a non-crystalline cefuroxime axetil solid dispersion or a substantially amorphous cefuroxime axetil.

4. The cefuroxime axetil granule composition of claim 1, which further comprises a coating material and a pharmaceutically acceptable additive.

5. The cefuroxime axetil granule composition of claim 4, wherein the coating material is an enteric coating material.

6. The cefuroxime axetil granule composition of claim 4, wherein the amounts of the coating material and the pharmaceutically acceptable additive are 0.2 to 10 and 0.02 to 50 parts by weight, respectively, based on 1 part by weight of a non-crystalline cefuroxime axetil solid dispersion or a substantially amorphous cefuroxime axetil.

7. A process for preparing a cefuroxime axetil granule having the composition of claim 1 comprising the steps of:
1) mixing the sucrose fatty acid ester and methacrylic acid-ethylacrylate copolymer, followed by melting the mixture with heating;
2) dispersing the disintegrating agent, and the non-crystalline cefuroxime axetil solid dispersion or substantially amorphous cefuroxime axetil in the molten mixture obtained in step 1); and
3) cooling the dispersion obtained in step 2), followed by pulverizing the cooled dispersion to obtain the granule.

8. The process of claim 7, wherein the melting temperature in step 1) is in the range from 60 to 75° C.
